(19) United States
(12) Patent Application Publication
(10) Pub. No.: US 2026/0248816 A1
(43) Pub. Date: Aug. 27, 2026

(54) Synergetic Formulation for Combination of Supplements

(71) Applicants: Jose Carlos Rocca, Doal, FL (US); David Jude Mastropietro, Davie, FL (US)

(72) Inventors: Jose Carlos Rocca, Doal, FL (US); David Jude Mastropietro, Davie, FL (US)

(21) Appl. No.: 19/063,538

(22) Filed: Feb. 26, 2025

(51) Int. Cl. A61K 31/593; A61K 31/122; A61K 31/191; A61K 33/06

(52) U.S. Cl. CPC A61K 31/593; A61K 31/122; A61K 31/191; A61K 33/06

(57) ABSTRACT

The present invention is a dosage form supplement wherein the ratio of Vitamin D3, Calcium β-Hydroxy-β-Methylbutyrate monohydrate (HMB), and Coenzyme Q₁₀ (CoQ) in a synergistic ratio w/w of Vit D:CoQ:HMB in a range of 1:1200-40000:120,000-320,000 is particular effective in mitigating muscle loss associated with rapid weight loss from bariatric surgery or GLP-1 drug use.

### BACKGROUND OF THE INVENTION

Rapid weight loss achieved through bariatric surgery or with the use of glucagon-like peptide-1 (GLP-1) receptor agonists, while beneficial for overall health, can have unintended consequences, most notably muscle loss. This loss of muscle mass can significantly impact various aspects of an individual's well-being, including:

Cardiovascular Health: Reduced muscle mass can negatively affect heart health by weakening the heart muscle and increasing the risk of heart disease.

Metabolic Health: Muscle mass plays a crucial role in regulating metabolism. Loss of muscle mass can lead to a decreased metabolic rate, making it more difficult to maintain weight loss and increasing the risk of metabolic disorders.

Muscle Performance & Physical Function: Muscle loss and reduced strength can greatly affect physical performance, resulting in decreased mobility, diminished strength, and a lower quality of life.

Given the critical importance of muscle mass for overall health and physical function, individuals utilizing GLP-1 medications and bariatric surgery for weight management will often require a modified nutritional approach to counteract muscle loss.

This invention addresses this need.

### SUMMARY OF THE INVENTION

The present invention provides a novel synergistic formulation with a specific and critical ratio relating to simultaneous administration of Vitamin D, Coenzyme Q10 (CoQ10), and Hydroxymethylbutyrate, also referred to as b-hydroxy b-methylbutyrate (HMB).

HMB (Beta-Hydroxy-Beta-Methylbutyrate): HMB is a metabolite of the amino acid leucine, known for its potent muscle-building and anti-catabolic properties. As used herein, HMB is identified by CAS 135236-72-5. It helps to prevent muscle breakdown and promotes muscle protein synthesis, crucial for maintaining and rebuilding muscle mass during weight loss. HMB of the present invention is suitable for use in an oral nutritional product and is compatible with the other essential elements of the product, including but not limited to: high bioavailability, good stability in the formulation, suitable for various dosage forms (tablets, capsules, powders), and pleasant taste and odor (if applicable). A specific embodiment uses a branded and clinically studied HMB (i.e., MyHMB®)

Vitamin D: Vitamin D is essential for normal muscle growth and development and plays a vital role in calcium absorption and bone health, both of which are essential for maintaining muscle strength and function. As used herein, Vitamin D is Cholecalciferol that is identified by CAS 67-97-0. It also has anti-inflammatory properties that can help to reduce muscle damage and inflammation associated with weight loss. It has also been shown to act synergistically with HMB to promote muscle health. A specific embodiment uses Vitamin D3 as cholecalciferol derived from lanolin. In another specific embodiment, the cholecalciferol may be derived from a vegan source including fungi (e.g., lichen, mushrooms), offering sustainable and ethical advantages.

Coenzyme Q10 (CoQ10): CoQ10 is an antioxidant that plays a crucial role in energy production within cells, particularly within the heart and muscles. As used herein, CoQ 10 is is identified by CAS 303-98-0. It helps to protect muscle cells from oxidative stress, improve mitochondrial function, and enhance cardiovascular health, which is particularly important during periods of rapid weight loss. Furthermore, CoQ10 in a stable solid dispersion (Phytosome®) delivery technology has been shown to reach and enhance intracellular concentrations of CoQ10 in cardiac cells compared to CoQ10 alone.

This synergistic combination of HMB, Vitamin D, and CoQ10 provides a comprehensive nutritional solution to mitigate muscle loss associated with rapid weight loss from bariatric surgery or GLP-1 drug use, thereby supporting overall health, physical function, and the long-term success of weight management programs.

In one embodiment, the present invention is a composition comprising:
Vitamin D3, as vegan cholecalciferol, Calcium β-Hydroxy-β-Methylbutyrate monohydrate (HMB), and Coenzyme Q₁₀ (CoQ) in a synergistic ratio w/w of Vit D:CoQ:HMB in a range of 1:1200-40000:120,000-320,000 in a dosage form with a nutraceutical carrier.

In one embodiment, the a synergistic ratio w/w of Vit D:CoQ:HMB is 1:2000-6666:20,000-53,000.

In one embodiment, the dosage form is an oral dosage form.

In one embodiment, the dosage form is an oral dosage tablet.

In one embodiment, the dosage form is a rapid dissolve oral dosage tablet.

In one embodiment, the dosage form is a coated oral dosage tablet.

In one embodiment, the dosage form is a composition consisting of:

Vitamin D3, as vegan cholecalciferol, Calcium β-Hydroxy-β-Methylbutyrate monohydrate (HMB), and Coenzyme Q₁₀ (CoQ) in a synergistic ratio w/w of Vit D:CoQ:HMB in a range of 1:1200-40000:120,000-320,000 in a dosage form with a nutraceutical carrier.

In one embodiment, the dosage form is an oral dosage form.

In one embodiment, the dosage form is an oral dosage tablet.

In one embodiment, the dosage form is a coated oral dosage tablet.

In one embodiment, the dosage form is a rapid dissolve oral dosage tablet.

In one embodiment, the dosage form is a composition consisting of:

Vitamin D3, as vegan cholecalciferol, Calcium β-Hydroxy-β-Methylbutyrate monohydrate (HMB), and Coenzyme Q₁₀ (CoQ) in a synergistic ratio w/w of Vit D:CoQ:HMB in a range of 1:2000-6666:20,000-53,000 in a dosage form with a nutraceutical carrier.

In one embodiment, the dosage form is an oral dosage form.

In one embodiment, the dosage form is an oral dosage tablet.

In one embodiment, the dosage form is a rapid dissolve oral dosage tablet.

In one embodiment, the dosage form is a coated oral dosage tablet.

### DETAILED DESCRIPTION OF THE INVENTION

As stated herein, the present invention provides a single dosage form whereby three components in a specific and critical ratio have been discovered to provide a unique synergistic effect to mitigate muscle loss associated with rapid weight loss from bariatric surgery or GLP-1 drug use.

The present invention is a composition comprising Vitamin D3 (as vegan cholecalciferol), Calcium β-Hydroxy-β-Methylbutyrate monohydrate (HMB), and Coenzyme Q10 (CoQ), wherein the ratio of Vitamin D3 to CoQ10 to HMB is expressed as the weight of the active ingredients (w/w) rather than the total weight of the raw materials used. The raw materials used to deliver these active components may include carriers, stabilizers, or salt forms that contribute additional weight but do not affect the specified ratio of active ingredients.

Although specific formulation are provided herein, these are merely illustrative. The impetus and novelty of the invention is the critical ratio of the three components that provide the desired mitigation of muscle loss associated with rapid weight loss from bariatric surgery or GLP-1 drug use.

### Example #1

This example illustrates an oral tablet formulation with a serving size of three (3) tablets, with each serving designed to provide 1,500 mg of calcium β-Hydroxy-β-Methylbutyrate monohydrate, 500 International Units (12.5 mcg) of vegan Vitamin D3 (derived from lichen), and 150 mg of CoQ 10 (as Ubiqsome®). The tablet formulation may include excipients such as microcrystalline cellulose and and magnesium stearate.

| Example #1 | mg | % |
|---|---|---|
| Calcium β-Hydroxy-β-Methylbutyrate | 500.00 | 61.61% |
| monohydrate | | |
| CoQ10 (as Ubiqsome ®) | 50.00 | 6.06% |
| Vitamin D3 (as vegan cholecalciferol) | 0.33 | 0.04% |
| Microcrystalline Cellulose | 266.14 | 32.26% |
| Magnesium Stearate | 8.50 | 1.03% |
| TOTAL | 824.97 | 100% |

### Example #2

This example illustrates an oral tablet formulation with a serving size of two (2) tablets, with each serving designed to provide 1,500 mg of calcium β-Hydroxy-β-Methylbutyrate monohydrate, with the preferred embodiment between 1.5 grams to 3 grams per 24 hours. 500 International Units (12.5 mcg) of Vitamin D3, and 150 mg of CoQ 10 (as Ubiqsome®). The tablet formulation may include excipients such as microcrystalline cellulose, croscarmellose sodium, and magnesium stearate.

| Example #2 | mg | % |
|---|---|---|
| Calcium β-Hydroxy-β-Methylbutyrate | 750.00 | 75.00% |
| monohydrate | | |
| CoQ10 (as Ubiqsome ®) | 50.00 | 5.00% |
| Vitamin D3 (as cholecalciferol) | 12.50 | 1.25% |
| Microcrystalline Cellulose | 132.50 | 13.25% |
| Croscarmellose Sodium | 33.00 | 3.30% |
| Magnesium Stearate | 22.00 | 2.20% |
| TOTAL | 1,000.0 | 100% |

These examples provide a method of administering a composition comprising an effective amount of Calcium β-Hydroxy-β-Methylbutyrate (HMB), CoQ10 Phytosome (e.g., Ubiqsome®), and Vitamin D3. The effective daily dosage of HMB is generally in the range of 1.5 to 4 grams, preferably 1.5 to 3 grams. The effective daily dosage of CoQ 10 Phytosome (e.g., Ubiqsome®), containing approximately 18% ubiquinone, is generally in the range of 150 to 500 milligrams, preferably 150 to 300 milligrams. The effective daily dosage of Vitamin D3 is generally in the range of 500 to 3,000 International Units (IU), preferably 2,000 to 3,000 IU (50 to 75 micrograms). The composition may be administered in one or more doses throughout the day. For example, the daily dose may be administered in 2-3 divided doses or as a single daily dose.

In one example, the tablets would have a cellulose-based (hydroxypropyl methylcellulose, hydroxypropyl cellulose) film-coating that is titanium dioxide-free. The film coating system examples are AquaPolish® F, manufactured by Biogrund, or Nutrafinish® by Colorcon®.

An example formula would be the coating system suspended in water up to a 15 percent solids level such as below:

| | | |
|---|---|---|
| Aquapolish | 15% | 150 g |
| Water | 85% | 850 g |
| TOTAL: | 100% | 1000 g |

This coating would be applied at an average weight gain of 2-6%

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

1. A composition comprising:
Vitamin D3, as vegan cholecalciferol, Calcium β-Hydroxy-β-Methylbutyrate monohydrate (HMB), and Coenzyme Q₁₀ (CoQ) in a synergistic ratio w/w of Vit D:CoQ:HMB in a range of 1:1200-40000:120,000-320,000 in a dosage form with a nutraceutical carrier.

2. The composition of claim 1, wherein said ratio is 1:2000-6666:20,000-53,000.

3. The composition of claim 1 wherein said dosage form is an oral dosage form.

4. The composition of claim 1 wherein said dosage form is an oral dosage tablet.

5. The composition of claim 1 wherein said dosage form is a rapid dissolve oral dosage tablet.

6. The composition of claim 1 wherein said dosage form is a coated oral dosage tablet.

7. A composition consisting of:
Vitamin D3, as vegan cholecalciferol, Calcium β-Hydroxy-β-Methylbutyrate monohydrate (HMB), and Coenzyme Q₁₀ (CoQ) in a synergistic ratio w/w of Vit D:CoQ:HMB in a range of 1:1200-40000:120,000-320,000 in a dosage form with a nutraceutical carrier.

8. The composition of claim 7 wherein said dosage form is an oral dosage form.

9. The composition of claim 7 wherein said dosage form is an oral dosage tablet.

10. The composition of claim 7 wherein said dosage form is a coated oral dosage tablet.

11. The composition of claim 7 wherein said dosage form is a rapid dissolve oral dosage tablet.

12. A composition consisting of:
Vitamin D3, as vegan cholecalciferol, Calcium β-Hydroxy-β-Methylbutyrate monohydrate (HMB), and Coenzyme Q₁₀ (CoQ) in a synergistic ratio w/w of Vit D:CoQ:HMB in a range of 1:2000-6666:20,000-53,000 in a dosage form with a nutraceutical carrier.

13. The composition of claim 12 wherein said dosage form is an oral dosage form.

14. The composition of claim 12 wherein said dosage form is an oral dosage tablet.

15. The composition of claim 12 wherein said dosage form is a rapid dissolve oral dosage tablet.

16. The composition of claim 12 wherein said dosage form is a coated oral dosage tablet.
